(19) United States
(12) Patent Application Publication
(10) Pub. No.: US 2026/0021290 A1
(43) Pub. Date: Jan. 22, 2026

(54) SYSTEMS AND METHODS FOR PROGRAMMING AND VISUALIZATION OF PERIPHERAL NERVE STIMULATION

(71) Applicant: Boston Scientific Neuromodulation Corporation, Valencia, CA (US)

(72) Inventors: Sarvani Grandhe, Valencia, CA (US); Chirag Shah, Santa Clarita, CA (US); Adarsh Jayakumar, Valencia, CA (US); Hari Hara Subramanian, Valencia, CA (US)

(21) Appl. No.: 19/270,194

(22) Filed: Jul. 15, 2025

Related U.S. Application Data
(60) Provisional application No. 63/672,566, filed on Jul. 17, 2024.

(51) Int. Cl. A61N 1/05; A61N 1/372

(52) U.S. Cl. CPC A61N 1/0556; A61N 1/37247

(57) ABSTRACT

A method or actions performed by a processor of a system includes displaying a representation of multiple tracts, fibers, nerve bundles, modulation targets, or symptoms, and a representation of electrodes of a cuff lead; in response to selection of at least one of the electrodes, displaying an indication of which tracts, fibers, or nerve bundles can be activated or which modulation targets or symptoms can be affected, using the at least one selected electrode; in response to selection of one of the tracts, fibers, nerve bundles, modulation targets, or symptoms, displaying an indication of at least one of the electrodes for activating the selected tract, fiber, or nerve bundle or for affecting the selected modulation target or symptom; and, optionally, programming a stimulator to stimulate the nerve using the at least one selected electrode or the at least one indicated electrode.

### CROSS-REFERENCE TO RELATED APPLICATIONS

This application claims the benefit under 35 U.S.C. § 119(e) of U.S. Provisional Patent Application Ser. No. 63/672,566, filed Jul. 17, 2024, which is incorporated herein by reference.

### FIELD

The present disclosure is directed to the area of peripheral nerve stimulation systems and methods of making and using the systems. The present disclosure is also directed to programming and visualization of stimulation using cuff devices.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a system for stimulating a nerve using a cuff lead disposed around the nerve, the cuff lead including a plurality of electrodes. The system includes a programming device including a display; a memory having instructions stored thereon; and a processor coupled to the display and the memory and configured to execute the instructions to perform actions. The actions include displaying, on the display, a representation of a plurality of tracts, fibers, nerve bundles, modulation targets, symptoms, or any combination thereof and a representation of the electrodes; in response to selection of at least one of the electrodes, displaying, on the display, an indication of which of the tracts, fibers, or nerve bundles can be activated or which of the modulation targets or symptoms can be affected, using the at least one selected electrode; in response to selection of a one of the tracts, fibers, nerve bundles, modulation targets, or symptoms, displaying, on the display, an indication of at least one of the electrodes for activating the selected one of the tracts, fibers, or nerve bundles or for affecting the selected one of the modulation targets or symptoms; and, optionally, programming a stimulator to stimulate the nerve using the at least one selected electrode or the at least one indicated electrode.

Another aspect is a method for stimulating a nerve using a cuff lead disposed around the nerve, the cuff lead including a plurality of electrodes. The method includes displaying, on a display, a representation of a plurality of tracts, fibers, nerve bundles, modulation targets, symptoms, or any combination thereof and a representation of the electrodes; in response to selection of at least one of the electrodes, displaying, on the display, an indication of which of the tracts, fibers, or nerve bundles can be activated or which of the modulation targets or symptoms can be affected, using the at least one selected electrode; in response to selection of a one of the tracts, fibers, nerve bundles, modulation targets, or symptoms, displaying, on the display, an indication of at least one of the electrodes for activating the selected one of the tracts, fibers, or nerve bundles or for affecting the selected one of the modulation targets or symptoms; and, optionally, programming a stimulator to stimulate the nerve using the at least one selected electrode or the at least one indicated electrode.

A further aspect is a non-transitory computer readable medium having instructions stored thereon, wherein the instructions, when executed using a processor, perform actions. The actions include displaying, on a display, a representation of a plurality of tracts, fibers, nerve bundles, modulation targets, symptoms, or any combination thereof and a representation of the electrodes; in response to selection of at least one of the electrodes, displaying, on the display, an indication of which of the tracts, fibers, or nerve bundles can be activated or which of the modulation targets or symptoms can be affected, using the at least one selected electrode; in response to selection of a one of the tracts, fibers, nerve bundles, modulation targets, or symptoms, displaying, on the display, an indication of at least one of the electrodes for activating the selected one of the tracts, fibers, or nerve bundles or for affecting the selected one of the modulation targets or symptoms; and, optionally, programming a stimulator to stimulate the nerve using the at least one selected electrode or the at least one indicated electrode.

In at least some of the aspects described above, each modulation target includes a body structure, the actions or method further including displaying, on the display, a plurality of the body structures located on a representation of a body; and, in response to selection of a one of the body structures, displaying, on the display, an indication of which of the electrodes can, by stimulating the nerve, produce a therapeutic effect at the selected one of the body structures.

In at least some of the aspects described above, the actions or method further include, in response to selection of the at least one of the electrodes and to obtaining at least a stimulation amplitude for each of the at least one of the electrodes, displaying, on the display, a representation of an estimated stimulation field or estimated charge distribution that can be generated using the selected at least one of the electrodes and the obtained simulation amplitude for each of the selected at least one of the electrodes.

In at least some of the aspects described above, the representation of the electrodes includes a representation of the cuff body unwrapped with the electrodes distributed thereon. In at least some of the aspects described above, the actions or method further include displaying, on the display, an image or graphical representation of the nerve.

In at least some of the aspects described above, the actions or method further include receiving a series of selections of at least one of the electrodes to define a temporal sequence of electrode activations to stimulate the nerve. In at least some of the aspects described above, the actions or method further include displaying, on the display, a time sequence of activations of the tracts, fibers, or nerve bundles according to the temporal sequence of electrode activations. In at least some of the aspects described above, the actions or method further include displaying, on the display, a time sequence of estimated stimulation fields or estimated charge distributions generated according to the temporal sequence of electrode activations.

In at least some of the aspects described above, displaying the indication of which of the tracts, fibers, or nerve bundles can be activated or which of the modulation targets or symptoms can be affected includes selecting the indication to identify a characteristic of a relationship between the at least one selected electrode and the indicated tracts, fibers, nerve bundles, modulation targets, or symptoms. In at least some of the aspects described above, the characteristic includes at least one of the following: a relative strength of the relationship, whether activation of the at least one selected electrode is beneficial or detrimental, or whether activation of the at least one selected electrode produces a neural activation effect or a neural blocking effect.

Yet another aspect is a system for stimulating a nerve using a cuff lead disposed around the nerve, the cuff lead including a plurality of electrodes. The system includes a programming device including a display; a memory having instructions stored thereon; and a processor coupled to the display and the memory and configured to execute the instructions to perform actions. The action includes obtaining a stimulation program including a temporal sequence of electrode activations, wherein each of the electrode activations includes at least one electrode that is selected to be activated and a stimulation amplitude for each of the selected at least one electrode, wherein at least two of the electrode activations differ in electrode selection, stimulation amplitude of at least one of the at least one selected electrode, or any combination thereof; displaying, on the display, a representation of the nerve and a representation of a plurality of the electrodes; displaying, on the representation of the nerve on the display, a time sequence of estimated stimulation fields; estimated charge distributions; activations of tracts, fibers, or nerve bundles; or any combination thereof corresponding to the temporal sequence of electrode activations; and, optionally, programming the stimulator to stimulate the nerve using the temporal sequence of electrode activations.

Another aspect is a method for stimulating a nerve using a cuff lead disposed around the nerve, the cuff lead including a plurality of electrodes. The method includes obtaining a stimulation program including a temporal sequence of electrode activations, wherein each of the electrode activations includes at least one electrode that is selected to be activated and a stimulation amplitude for each of the selected at least one electrode, wherein at least two of the electrode activations differ in electrode selection, stimulation amplitude of at least one of the at least one selected electrode, or any combination thereof; displaying, on a display, a representation of the nerve and a representation of a plurality of the electrodes; displaying, on the representation of the nerve on the display, a time sequence of estimated stimulation fields; estimated charge distributions; activations of tracts, fibers, or nerve bundles; or any combination thereof corresponding to the temporal sequence of electrode activations; and, optionally, programming the stimulator to stimulate the nerve using the temporal sequence of electrode activations.

A further aspect is a non-transitory computer readable medium having instructions stored thereon, wherein the instructions, when executed using a processor, perform actions. The actions include obtaining a stimulation program including a temporal sequence of electrode activations, wherein each of the electrode activations includes at least one electrode that is selected to be activated and a stimulation amplitude for each of the selected at least one electrode, wherein at least two of the electrode activations differ in electrode selection, stimulation amplitude of at least one of the at least one selected electrode, or any combination thereof; displaying, on a display, a representation of the nerve and a representation of a plurality of the electrodes; displaying, on the representation of the nerve on the display, a time sequence of estimated stimulation fields; estimated charge distributions; activations of tracts, fibers, or nerve bundles; or any combination thereof corresponding to the temporal sequence of electrode activations; and, optionally, programming the stimulator to stimulate the nerve using the temporal sequence of electrode activations.

In at least some of the aspects described above, the actions or method further include displaying, on the display, the temporal sequence of electrode activations. In at least some of the aspects described above, displaying the temporal sequence of electrode activations includes connecting individual representations of the electrodes according to the temporal sequence. In at least some of the aspects described above, displaying the temporal sequence of electrode activations includes displaying, for each of multiple electrodes of the plurality of the electrodes, a graph of electrode activation or stimulation amplitude versus time according to the temporal sequence.

In at least some of the aspects described above, obtaining the temporal sequence includes receiving user input of stimulation parameters to define the temporal sequence of electrode activations. In at least some of the aspects described above, displaying the time sequence includes displaying the time sequence as an animation or video.

Yet another aspect is a system for sensing a nerve using a cuff lead disposed around the nerve, the cuff lead including a plurality of electrodes. The system includes a programming device including a display; a memory having instructions stored thereon; and a processor coupled to the display and the memory and configured to execute the instructions to perform actions. The action include displaying, on the display, a representation of a plurality of tracts, fibers, or nerve bundles of the nerve and a representation of at least one or more of the electrodes; in response to selection of at least one of the electrodes, displaying, on the display, an indication of which of the tracts, fibers, or nerve bundles can be sensed using the selected at least one of the electrodes; in response to selection of a one of the tracts, fibers, or nerve bundles, displaying, on the display, an indication of at least one of the electrodes for sensing the selected one of the tracts, fibers, or nerve bundles; and, optionally, programming the stimulator to sense the nerve using the at least one selected electrode or the at least one indicated electrode.

Another aspect is a method for sensing a nerve using a cuff lead disposed around the nerve, the cuff lead including a plurality of electrodes. The method includes a programming device including a display; a memory having instructions stored thereon; and a processor coupled to the display and the memory and configured to execute the instructions to perform actions. The action include displaying, on a display, a representation of a plurality of tracts, fibers, or nerve bundles of the nerve and a representation of at least one or more of the electrodes; in response to selection of at least one of the electrodes, displaying, on the display, an indication of which of the tracts, fibers, or nerve bundles can be sensed using the selected at least one of the electrodes; in response to selection of a one of the tracts, fibers, or nerve bundles, displaying, on the display, an indication of at least one of the electrodes for sensing the selected one of the tracts, fibers, or nerve bundles; and, optionally, programming the stimulator to sense the nerve using the at least one selected electrode or the at least one indicated electrode.

A further aspect is a non-transitory computer readable medium having instructions stored thereon, wherein the instructions, when executed using a processor, perform actions. The actions includes a programming device including a display; a memory having instructions stored thereon; and a processor coupled to the display and the memory and configured to execute the instructions to perform actions. The action include displaying, on a display, a representation of a plurality of tracts, fibers, or nerve bundles of the nerve and a representation of at least one or more of the electrodes; in response to selection of at least one of the electrodes, displaying, on the display, an indication of which of the tracts, fibers, or nerve bundles can be sensed using the selected at least one of the electrodes; in response to selection of a one of the tracts, fibers, or nerve bundles, displaying, on the display, an indication of at least one of the electrodes for sensing the selected one of the tracts, fibers, or nerve bundles; and, optionally, programming the stimulator to sense the nerve using the at least one selected electrode or the at least one indicated electrode.

In at least some of the aspects described above, the actions or method further include displaying, on the display, an image or graphical representation of the nerve. In at least some of the aspects described above, the actions or method further includes receiving a series of selections of at least one of the electrodes to define a temporal sequence of electrode sensing of the nerve.

In at least some aspects, any of the systems described above further includes the cuff lead including a cuff body configured for disposition around the nerve with the plurality of electrodes disposed on the cuff body and the stimulator coupled or coupleable to the cuff lead and configured to generate stimulation signals and deliver the stimulation signals through at least one of the electrodes to stimulate the nerve.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to a control module;

FIG. 2A is a schematic view of one embodiment of the control module of FIG. 1 configured and arranged to electrically couple to an elongated device;

FIG. 2B is a schematic view of one embodiment of a lead extension configured and arranged to electrically couple the elongated device of FIG. 2A to the control module of FIG. 1;

FIG. 3 is a schematic perspective view of one embodiment of a cuff with two sets of sixteen longitudinal electrodes each and two radial electrodes;

FIG. 4 is a flowchart of one embodiment of a method for stimulating a nerve using a cuff lead;

FIG. 5 is a cross-sectional view of a cuff disposed around a portion of a nerve;

FIG. 6 is a view of one embodiment of a display or interface with a representation of electrodes of a cuff lead and a representation of tracts, fibers, nerve bundles, modulation targets, or symptoms that can be affected by stimulation of a nerve using the cuff lead;

FIG. 7 is a cross-sectional view of a cuff disposed around a portion of a nerve illustrating an estimated stimulation field or charge distribution;

FIG. 8 is a flowchart of another embodiment of a method for stimulating a nerve using a cuff lead;

FIG. 9 is a view of one embodiment of a display or interface for indicating a temporal sequence of electrode activations;

FIG. 10 is a view of another embodiment of a display or interface for indicating a temporal sequence of electrode activations;

FIG. 11 is a flowchart of one embodiment of a method for sensing a nerve using a cuff lead; and

FIG. 12 is a schematic overview of one embodiment of components of an electrical stimulation arrangement according to an embodiment of the present invention.

### DETAILED DESCRIPTION

The present disclosure is directed to the area of peripheral nerve stimulation systems and methods of making and using the systems. The present disclosure is also directed to programming and visualization of stimulation using cuff devices.

Suitable implantable electrical stimulation systems include, but are not limited to, a least one lead with one or more electrodes disposed along a distal end of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734; 7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent application Ser. Nos. 12/177,823 and 13/750,725, all of which are incorporated by reference in their entireties.

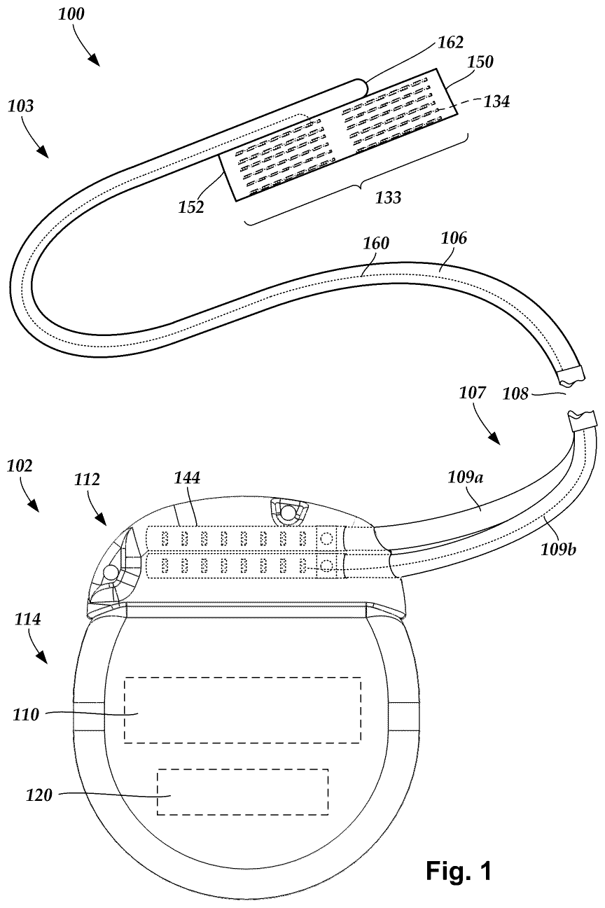

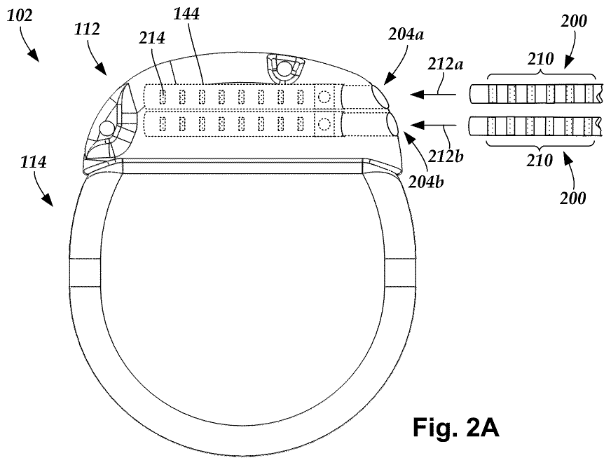

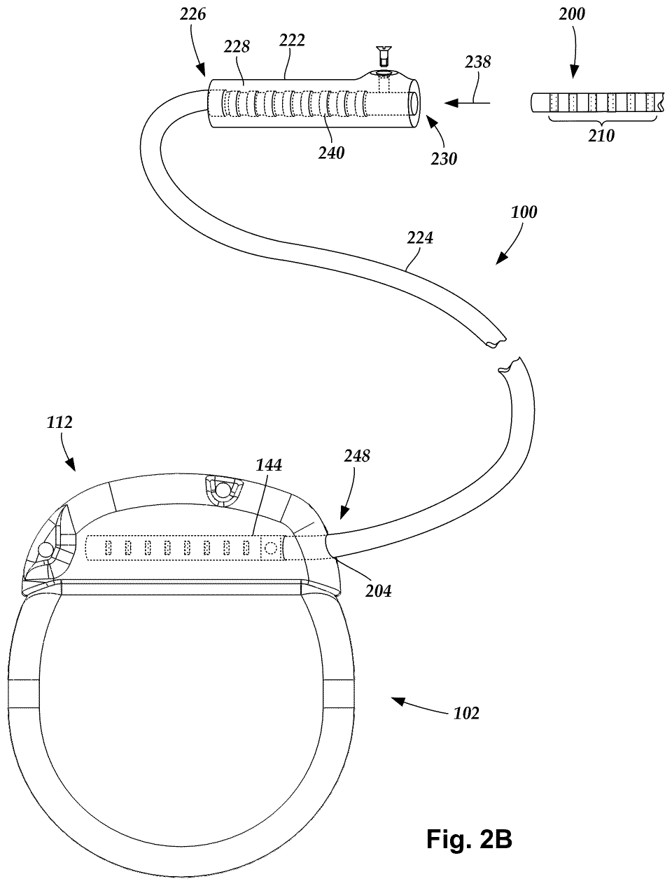

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a mount 162 and a cuff 150 with a cuff body 150 and an array of electrodes 133, such as electrode 134. The lead 103 also includes one or more lead bodies 106, coupled to or containing the mount 162, and an array of terminals (e.g., 210 in FIG. 2A-2B) attached to the one or more lead bodies 106. In at least some embodiments, the lead is isodiametric along at least a portion of the longitudinal length of the lead body 106. FIG. 1 illustrates one lead 103 coupled to a control module 102. Other embodiments may include two, three, four, or more leads 103 coupled to the control module 102. In yet other embodiments, a lead 103 may be coupled to multiple control modules 102. For example, a lead with 64 electrodes may be coupled to two control modules 102 that are capable of handling 32 electrodes each.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 1, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more proximal tails 109a and 109b configured and arranged to couple to the control module 102 (or another splitter, a lead extension, an adaptor, or the like). The splitter 107 and splitter connector 108 may be part of the lead 103 or may be a separate component that attaches to the lead.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the lead 103 and the stimulation circuitry 110 of the control module 102.

The electrical stimulation system or components of the electrical stimulation system, including the lead body 106 and the control module 102, are typically implanted into the body of a patient. The electrical stimulation system can be used for a variety of applications including, but not limited to, brain stimulation, neural stimulation, spinal cord stimulation, muscle stimulation, and the like.

The lead body 106 can be made of, for example, a non-conductive, biocompatible material such as, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end of the lead body 106 to the proximal end of the lead body 106.

Terminals (e.g., 210 in FIGS. 2A-2B) are typically disposed along the proximal end of the lead body 106 of the electrical stimulation system 100 (as well as any splitters, lead extensions, adaptors, or the like) for electrical connection to corresponding connector contacts (e.g., 214 and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires 160, cables, or the like (only one of which is shown in FIG. 1) extend from the terminals to the electrodes 134. Typically, one or more electrodes 134 are electrically coupled to each terminal. In at least some embodiments, each terminal is only connected to one electrode 134.

The electrically conductive wires (“conductors”) 160 (only one of which is illustrated in FIG. 1 for clarity) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

FIG. 1 also illustrates a mount 162, part of the lead body 106, coupled to cuff 150. The conductors 160 (only one of which is illustrated in FIG. 1 for clarity) from within the lead body 106 are received in the mount 162, which in turn is attached to the cuff 150 such that each conductor passes through the mount 162 for a direct electrical connection with one of the electrodes 134 (e.g., one conductor is electrically connected with one electrode and so on). The mount 162 may be attached using a variety of means such as, but not limited to, molding or adhering the mount 162 to the cuff 150. In other embodiments, the conductors 160 from within the lead body 106 are electrically coupled to the electrodes 134 using jumper, intermediate or transition wires from the lead body 106 to the electrodes 134.

The mount 162 can be offset from the cuff 150, as illustrated in FIG. 1, or in-line with the cuff or in any other suitable arrangement. Non-limiting examples of cuff leads 103 can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0226641; and 2022/0370793 and U.S. Provisional Patent Application Ser. Nos. 63/539,774 and 63/549,797, all of which are incorporated herein by reference in their entireties.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the lead extension 224 of FIG. 2B, an adaptor, or the like or combinations thereof), or a combination thereof. FIG. 2A illustrates two elongated devices 200 coupled to the control module 102. These two elongated devices 200 can be two tails as illustrated in FIG. 1 or two different leads or any other combination of elongated devices.

The control module connector 144 defines at least one port into which a proximal end of the elongated device 200 can be inserted, as shown by directional arrow 212. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. Nos. 7,244,150 and 8,224,450, which are incorporated by reference in their entireties.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

Any suitable arrangement of electrodes 134 on the cuff 150 can be used. Examples of cuff leads 103 and electrode arrangements for cuff leads can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0370793; and 2022/0395690; and U.S. Provisional Patent Application Ser. Nos. 63/539,774 and 63/549,797, all of which are incorporated herein by reference in their entireties.

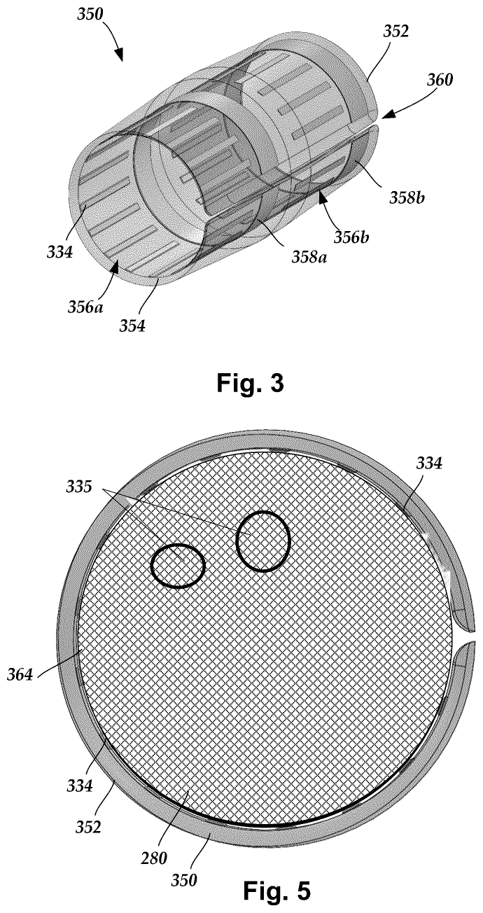

FIG. 3 illustrates one embodiment of a cuff 350 of a cuff lead 103 (FIG. 1). The cuff 350 includes a cuff body 352 with longitudinal electrodes 334 disposed on an interior surface 354 of the cuff body and arranged around the circumference of the cuff body in two sets 356a, 356b. In the illustrated embodiment, each set 356a, 356b includes sixteen longitudinal electrodes 334. Any other suitable number of electrodes can be used including, but not limited to, 16, 20, 25, 28, 32, 36, 40, 48, 50, 64, 80, 100, 120, 128, 150, 200, 250, 256, or more longitudinal electrodes. A cuff lead can have one, two, three, four, or more sets of longitudinal electrodes 334. The number of longitudinal electrodes 334 in a set can be the same for each set or can differ. In the illustrated embodiment, the longitudinal electrodes 334 of each set are aligned longitudinally with electrodes of the other set. In other embodiments, the longitudinal electrodes 334 of each set can be staggered or unaligned with the electrodes of the other set.

In addition, the cuff 350 includes two radial electrodes 358a, 358b that wrap around at least 75%, 80%, 90%, or 95% of the circumference of the cuff body 352. The cuff 350 also defines a slit 360 that extends the longitudinal length of the cuff body 352 so that the nerve can be loaded into the interior 362 of the cuff body by opening the slit to fit the cuff body over the nerve. The slit 360 is opened or initially sized to allow the target nerve (not shown) to be slipped, inserted, fed, or otherwise received into the cuff 350 such that the cuff 350 wraps around the target nerve. In at least some embodiments, the slit 360 allows the cuff 350 to be easily moved over and around the target nerve or relative to the target nerve whether rotationally or transitionally.

The electrodes 134, 334, 358a, 358b can be formed using any conductive, biocompatible material. Examples of suitable materials include metals, alloys, conductive polymers, conductive carbon, and the like, as well as combinations thereof. In at least some embodiments, one or more of the electrodes 134, 334, 358a, 358b are formed from one or more of: platinum, platinum alloys such as platinum iridium, palladium alloys such as palladium rhodium, titanium, titanium alloys, nickel alloys, cobalt alloys, nickel/cobalt alloys, stainless steel, tantalum, conductive carbon, conductive plastics, epoxy or other adhesive filled with metallic powder, Nitinol™, or the like or any combination thereof. The electrodes 134, 334, 358a, 358b can be formed by any suitable process including, but not limited to, machining, molding (for example, powdered metal molding), photolithography, additive techniques, stamping, or the like or any combination thereof.

The cuff body 152, 352 can be formed of any suitable biocompatible and biostable non-conductive material including, but not limited to, polymer materials such as silicone, polyurethane, polyetheretherketone (“PEEK”), epoxy, or the like or any combination thereof. In at least some embodiments, the cuff body 352 can have a circular, oval, or any other suitable cross-sectional shape and, at least in some embodiments, may be sufficiently flexible to alter the cross-sectional shape to accommodate the nerve. In at least some embodiments, the electrodes 134, 334, 358a, 358b can be molded with the cuff body 152, 352 or formed by techniques such as etching or ablation of conductive layers, films, or the like. In at least some embodiments, the cuff body 152, 352 has an inner diameter (which can correspond to the largest diameter of a non-circular cuff body) in a range of 0.5 to 5 mm or in a range of 1 to 3 mm. In at least some embodiments, the cuff body 152, 352 has a length of at least 5, 10, or 20 mm.

In at least some embodiments, the cuff body 152, 352 can be formed using any suitable technique including, but not limited to, molding, casting, formed in a sheet and then shaped using adhesive as a binder, formed flat and shaped using heat, formed flat and attached to a cuff-shaped scaffold, pressed or extruded into the cuff shape, or the like or any combination thereof. In at least some embodiments, the electrodes 134, 334, 358a, 358b can be attached to the cuff body 152, 352 using any suitable technique including, but not limited to, attaching with adhesive, molding (for example, insert molding) into the cuff body, using heat to adhere the electrodes to the cuff body, heating and pressing the electrodes into the cuff body, depositing electrode material on the cuff body and using photolithography and etching, or the like or any combination thereof.

In at least some embodiments, once the cuff 350 has been placed in a desired position relative to the target nerve, the edges of the cuff body 352 defining the slit 360 can be sutured to capture the target nerve without undesirably compressing the target nerve. In at least some embodiments, suture holes (not shown) are optionally incorporated into the edges of the cuff 350 to allow for closing or partially closing the cuff 350 around the target nerve. Any other suitable arrangement, method, or technique can be used to secure the cuff 350 to the target nerve.

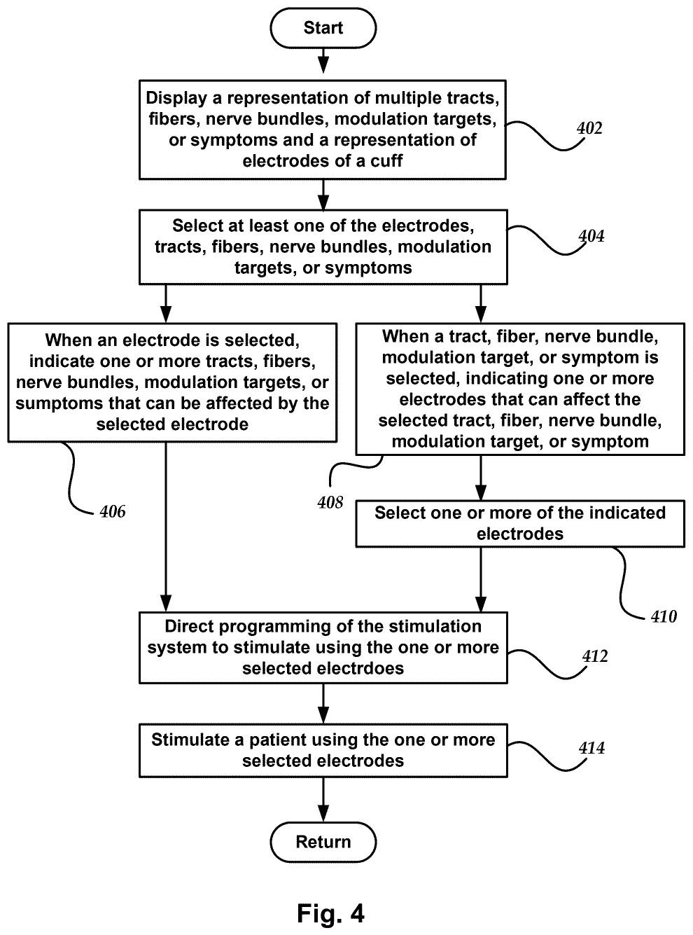

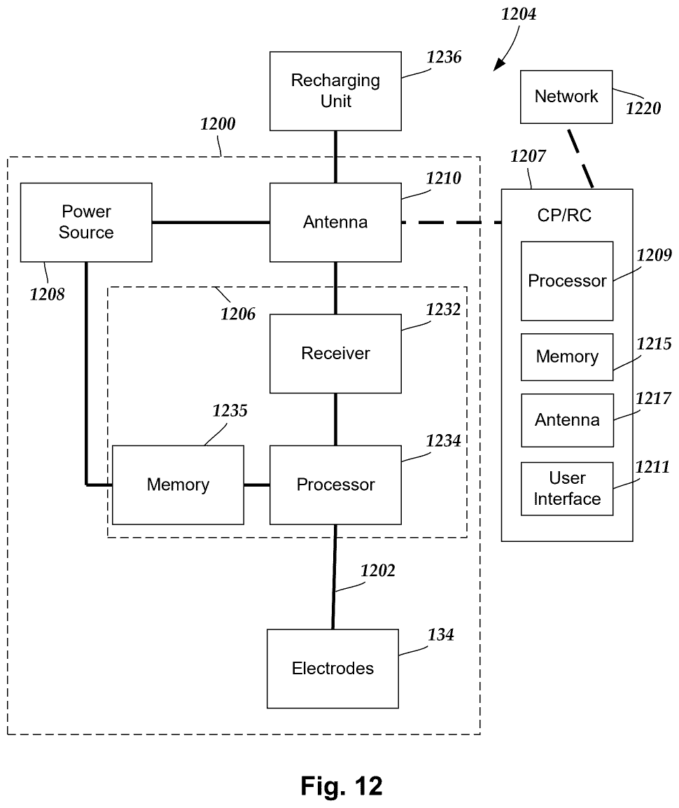

FIG. 4 is a flow chart of one embodiment of a method for selecting stimulation electrodes for stimulation of a nerve of a patient using a cuff lead. In step 402, a representation of multiple tracts, fibers, nerve bundles, modulation targets, symptoms, or any combination thereof and a representation of the electrodes 334, 358a, 358b of a cuff 350 of a cuff lead (e.g., cuff lead 103 (FIG. 1)) is displayed (for example, on a display of a remote control, clinician programmer, or other suitable device or computer system, such as the system illustrated in FIG. 12 or devices/systems described in the reference cited herein).

For example, FIG. 5 illustrates one embodiment of the displayed representations in the form of a cross-section of a cuff 350 with electrodes 334 disposed around a nerve 280 (e.g., the vagus nerve). The electrodes 334 are arranged around the circumference of the cuff 350 and nerve 280. In at least some embodiments, one or more tracts, fibers, or nerve bundles 335 (or other physiological elements) of the nerve 280 can be identified in the displayed representation of the nerve, as illustrated in FIG. 5.

The representations can include an image or any other suitable graphical representation (e.g., FIG. 5) of the nerve 280. The image can be any suitable image including, for example, a computer tomography (CT) image, a magnetic resonance image (MRI), or a functional magnetic resonance image (FMRI) or any combination thereof. In at least some embodiments, the graphical representation of the nerve 280 can be based on image, a generic representation of a generic or specific nerve, or any other suitable representation of the nerve. In at least some embodiments, the image or graphical representation of the nerve 280 provides anatomical or structural information about the nerve 280, such as, the position of one or more tracts, fibers, or nerve bundles 335. The image can be an image from the patient or an image from another source, such as another patient or an anatomical atlas. It will be understood that a display or interface can, instead, display a drawing of the nerve with tracts, fibers, or nerve bundles 335 or any other suitable representation.

The orientation (e.g., the rotational orientation (around the circumference of the nerve) or longitudinal orientation (along the length of the nerve)) of the electrodes 334 relative to the nerve 280 and tracts, fibers, or nerve bundles 335 in the nerve can be determined or estimated by any suitable method of technique. For example, the orientation can be determined or estimated based on observation during implantation, testing of electrodes to identify stimulation effects related to the respective electrodes, or the like or any combination thereof.

The display or interface in FIG. 5 includes a representation of the cuff body 352, but the cuff body can be removed in other embodiments. The representations of the individual electrodes can be labeled (e.g., E1, E2, E3, . . . ). If there are multiple sets of electrodes, such as the two sets 356a, 356b of electrodes 334 of the cuff 350 of FIG. 3, there can be multiple displays or interfaces of different portions of the cuff 350 and the nerve 280 such as, for example, at least one display or interface for each of the sets 356a, 356b. Multiples displays or interfaces at different points along the nerve 280 can be provided for any cuff 350. This set of displays or interfaces can provide a three-dimensional spatial map of the nerve and cuff 350/electrodes 334. In at least some embodiments, a user can select any one of the displays or interfaces to view in detail.

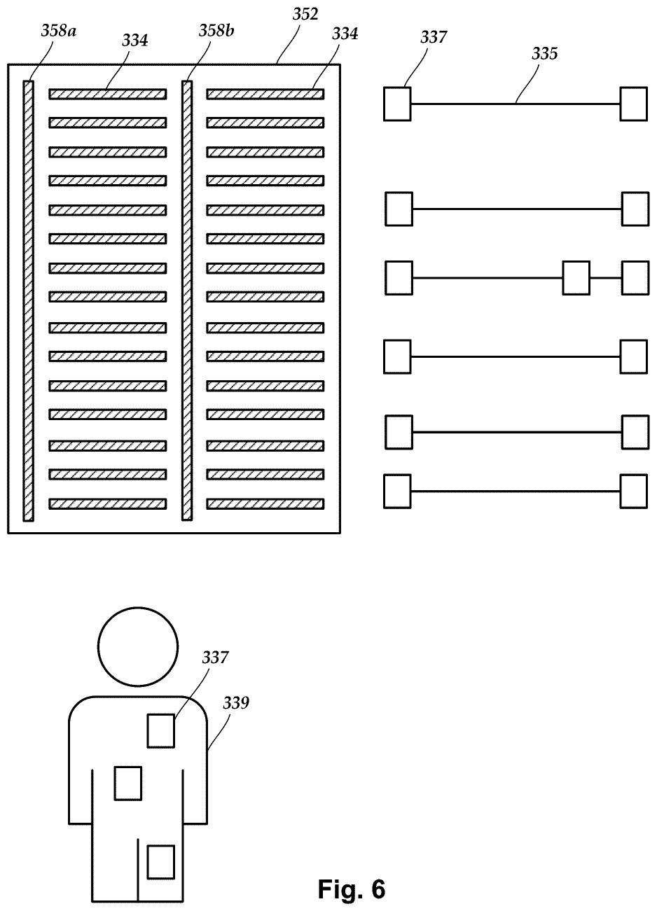

FIG. 6 illustrates another display or interface with a representation of a cuff 350 with electrodes 334, 358a, 358b. The cuff is unwrapped and laid flat for illustration or user interaction (for example, for user selection of one or more electrodes). FIG. 6 also includes graphical representations (for example, lines, bars, arrows, or the like) of one or more tracts, fibers, or nerve bundles 335. In at least some embodiments, the representations of the tracts, fibers, or nerve bundles 335 can be displayed with indications of one or more body structures 337 (e.g., organs, brain structures, other nerves, or tissue or the like or any combination thereof) at the ends. One or more symptoms or stimulation effects (e.g., therapeutic effects, side effects, or any other effects) can be displayed with, or as an alternative, to the body structure(s). (Reference to “the body structure(s) 337” herein can be substituted by, or accompanied by, “the symptoms” or “the stimulation effect(s)”.) In at least some embodiments, an indication of one or more body structures 337 can be placed along the length of at least one or more of the tracts, fibers, or nerve bundles 335. Each indication can represent one or more of the body structures 337, symptoms, or stimulation effects. The body structures 337, symptoms, or stimulation effects represented by the indications can be modulation targets that can be affected (for example, positively or negatively) by stimulation of the nerve 280 that the cuff 350 is attached to.

In at least some embodiments, the representations of the electrodes 334 on the display or interface are aligned with the representation of the corresponding tracts, fibers, or nerve bundles 335 or body structures 337 that can be affected by activation of the aligned electrode. FIG. 6 also includes an optional (or alternative) representation of a body 339 with indications of the body structures 337. At least some of those body structures 337 can be affected when one or more of the electrodes 334 are activated.

Any other suitable arrangement of any combination of the electrodes 334, 358a, 358b; the cuff 350; the tracts, fibers, or nerve bundles 335; or the body structures 337 can be used. In at least some embodiments, a stimulation system can allow a user to select from different arrangements, remove or add one or more of these elements from the arrangement, create a particular arrangement of these elements, or the like or any combination thereof.

In at least some embodiments, when the representation(s) of one or more electrodes 334, 358a, 385b is highlighted or selected, one or more of the tracts, fibers, or nerve bundles 335 or body structures 337, which are affected by activation of the selected or highlighted electrode(s), are highlighted or otherwise indicated. In at least some embodiments, when the representation(s) of one or more of the tracts, fibers, or nerve bundles 335 or body structures 337 are highlighted or selected, one or more electrodes 334, 358a, 385b, which can affect the selected or highlighted tracts, fibers, nerve bundles, or body structures, are highlighted or otherwise indicated. This responsive highlighting or indicating identifies a relationship between the highlighted (or otherwise indicated) electrode(s) 334, 358a, 385b and the highlighted (or otherwise indicated) tracts, fibers, or nerve bundles 335 or body structures 337. Any other suitable technique or graphical representation for indicating relationships between the electrodes 334, 358a, 358b and the tracts, fibers, or nerve bundles 335 or body structures 337 that can be affected when the electrode(s) is activated.

In at least some embodiments, the indication of a relationship may depend upon the value(s) for one or more stimulation parameters, such as, for example, stimulation amplitude (total or for individual electrodes), pulse width, stimulation duration, or the like or any combination thereof. For example, a selection of a stimulation amplitude at or above a minimum amplitude is needed to indicate the relationship.

Any suitable mechanism, method, or technique (or combination thereof) can be used to determine whether a relationship exists between one or more electrode(s) 334, 358a, 385b and one or more tracts, fibers, or nerve bundles 335 or body structures 337. For example, a relationship can be determined based on observations (for example, by a user, clinician, or other individual or using a sensor or other device), measurements (e.g., using one or more sensors), results or effects of test stimulations on one or more tracts, fibers, or nerve bundles 335 or body structures 337 using one or more electrode(s) 334, 358a, 385b, or the like or any combination thereof. In at least some embodiments, the test stimulations can be performed on the patient. In at least some embodiments, the test stimulations have been performed on other patients and then mapped onto the electrodes 334, 358a, 385b of a cuff 350 disposed around the nerve of the patient. Non-limiting examples of methods for obtaining and recording clinical effects can be found at, for example, U.S. Pat. Nos. 9,227,074; 9,248,296; 9,358,398; 9,474,903; 10,071,249; 10,357,657; 10,369,364; 10,603,498; and 10,716,505; U.S. Patent Application Publications Nos. 2014/0243926; 2014/0276707; 2014/0277282; 2014/0277284; 2018/0264278; 2020/0376263; 2020/0398057; 2021/0023374; 2023/0181090; and 2024/0157151; and U.S. patent application Ser. No. 18/535,655, all of which are incorporated herein by reference in their entireties.

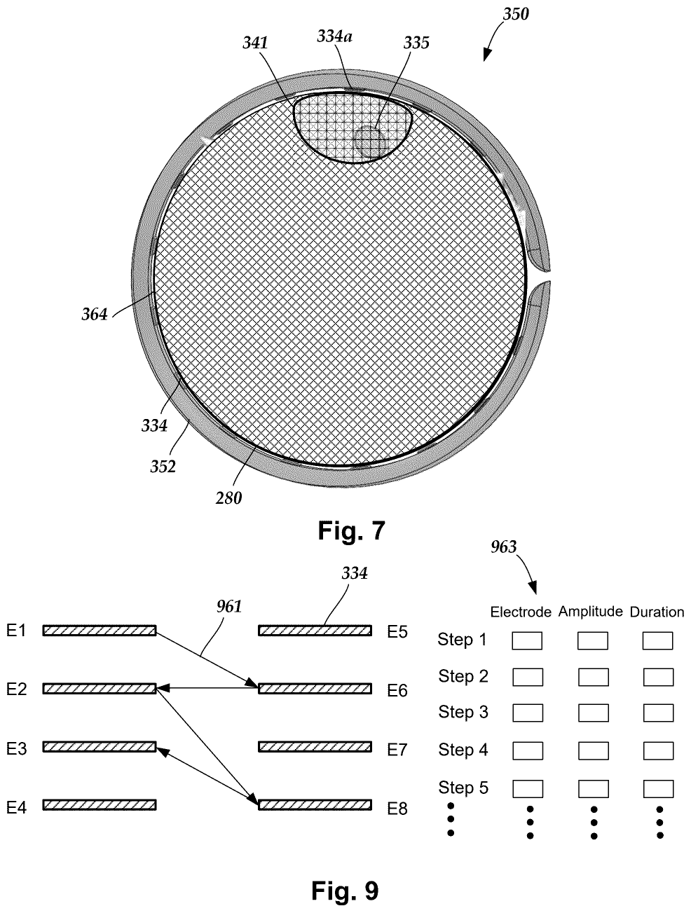

In at least some embodiments, the relationship between one or more electrode(s) 334, 358a, 385b and one or more tracts, fibers, or nerve bundles 335 or body structures 337 can be determined by estimating a stimulation field, charge distribution, center of stimulation, or the like. FIG. 7 illustrates one embodiment of a cuff 350 with electrodes 334 disposed around a nerve 280. A stimulation field 341 (e.g., volume of activation (VOA), volume of tissue activated (VTA), stimulation field map (SFM), charge distribution, center of stimulation, or the like) generated using one or more electrodes (for example, electrode 334a) can be estimated and compared to the position of a tract, fiber, or nerve bundle 335, as illustrated in FIG. 7. The extent or size of the stimulation field 341 can depend on the value(s) of one or more other stimulation parameters, such as, for example, stimulation amplitude (total or for individual electrodes), pulse width, stimulation duration, or the like. In at least some embodiments, variation of the stimulation field or charge distribution can be depicted using any suitable graphical arrangement including, but not limited to, a heat map, a contour map, or a cluster map. A user can specify one or more of these stimulation parameters, in addition to a selection of one or more electrodes 334, 358a, 358b; tracts, fibers, or nerve bundles 335; or body structures 337. Non-limiting examples of methods for estimating a stimulation field (e.g., volume of activation, volume of tissue activated, charge distribution, or the like) can be found at U.S. Pat. Nos. 8,326,433; 8,675,945; 8,831,731; 8,849,632; 8,958,615; and 10,265,528; U.S. Patent Application Publications Nos. 2009/0287272; 2009/0287273; 2012/0314924; 2013/0116744; 2014/0122379; and 2015/0066111, all of which are incorporated herein by reference in their entireties.

In at least some embodiments, the indication of the relationship between a particular electrode 334, 358a, 385b (or group of electrodes) and each of one or more tracts, fibers, or nerve bundles 335 or body structures 337 can also indicate one or more characteristics of the relationship including, but not limited to, the relative strength of the relationship, whether the electrode activation is beneficial (e.g., a therapeutic effect) or detrimental (e.g., a side effect); whether the electrode activation produces a neural activation effect or a neural blocking effect; or the like or any combination thereof. In at least some embodiments, one or more of the characteristics can also depend on one or more other stimulation parameters, such as, for example, amplitude (total or specific to an electrode), pulse width, pulse frequency, duration of electrode activation, or the like or any combination thereof. Any suitable method for indicating the relationship can be used including, but not limited to, different colors, different brightness, different graphical shapes or other graphical indicia, or the like or any combination thereof. In at least some embodiments, one or more of these characteristics may depend upon a selection of one or more stimulation parameters, such as, for example, stimulation amplitude (total or for individual electrodes), pulse width, stimulation duration, or the like or any combination thereof.

In step 404, a user selects one (or more) of the electrodes 334, 358a, 358b; tracts, fibers, or nerve bundles 335; body structures 337; or symptoms. The method proceeds to either step 406 or 408 depending on the selection. Any suitable method can be used for making a selection can be used including, but not limited to, selection from a menu; point-and-click selection (or the like); alphanumerical entry of a designation for the electrode, tract, fiber, nerve bundle, or body structure; entry or selection of a symptom, disease, disorder, therapeutic effect, or the like followed by association with one or more tracts, fibers, nerve bundles, or body structures, or the like or any combination thereof.

In step 406, when the user selects one (or more) of the electrodes 334, 358a, 358b, any of the tracts, fibers, or nerve bundles 335; body structures 337; or symptoms that are affected by the selected electrode(s) are indicated on the display or interface based on the relationship between the electrode(s) and the tracts, fibers, nerve bundles; body structures; or symptoms. It will be understood that the method can return to step 404 for another user selection.

In step 408, when the user selects one (or more) of the tracts, fibers, or nerve bundles 335; body structures 337; or symptoms, any of the electrodes 334, 358a, 358b that can affect the selected tract(s), fiber(s), nerve bundle(s), body structure(s); or symptom(s) are indicated on the display or interface based on the relationship between the electrode(s) and the tracts, fibers, nerve bundles, body structures, or symptoms. It will be understood that the method can return to step 404 for another user selection. In step 410, the user selects one or more of the indicated electrodes 334, 358a, 358b for stimulation.

After step 406 or 410, in step 412, the user directs (or the system automatically initiates) programming of the stimulation system to stimulate the patient using the one or more selected electrodes. In at least some embodiments, the stimulation program is also programmed with one or more additional stimulation parameters that have been chosen, chosen by the user when queried, or automatically or default selected. In step 414, the stimulation system stimulates the patient using the one or more selected electrodes.

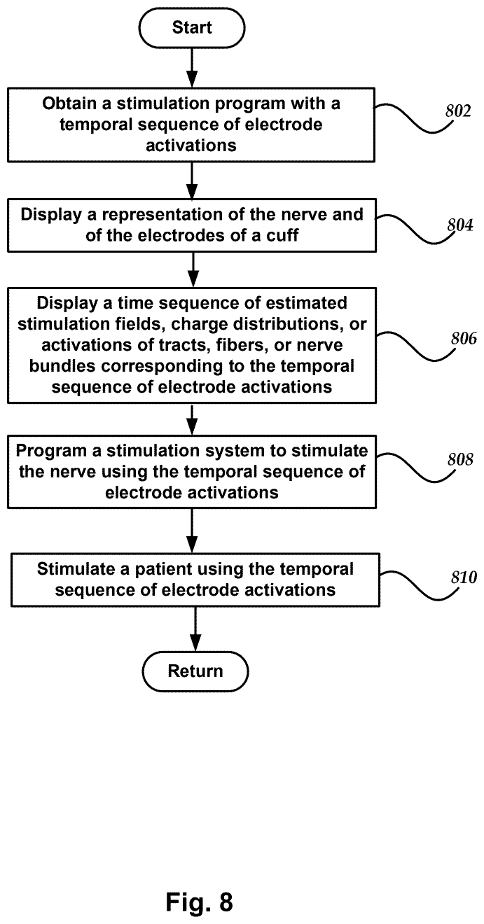

FIG. 8 is a flow chart of another embodiment of a method for selecting stimulation electrodes for stimulation of a nerve of a patient using a cuff lead. In step 802, a stimulation program having a temporal sequence of electrode activations is obtained. In at least some embodiments, the stimulation program is obtained from a programmed control module, a remote control device, a clinician programmer, a database of stimulation programs, or any other suitable source. In at least some embodiments, the stimulation program is obtained by user selection of the electrodes and other stimulation parameters of the temporal sequence. Any other suitable method for obtaining the stimulation program can be used. At least two of the electrode activations are different from each other in electrode selection, stimulation amplitude of at least one electrode, pulse width, or the like or any combination thereof.

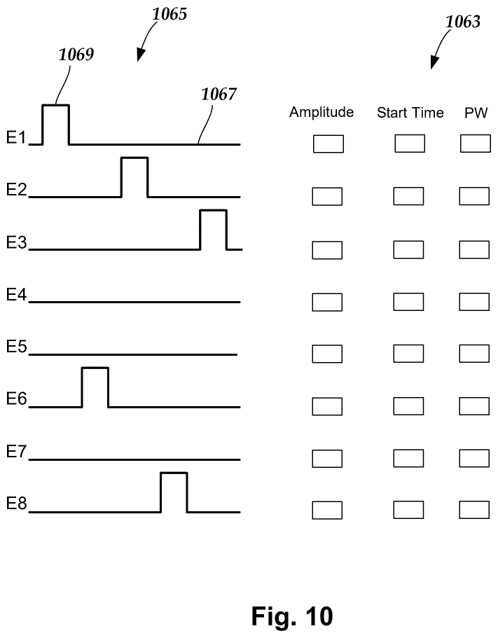

FIG. 9 is an illustration of one embodiment of a display or interface for defining or illustrating the temporal sequence of electrode activations. This interface includes a representation of the electrodes 334 of a cuff (e.g., cuff 350 of FIG. 3). In at least some embodiments, a user can select individual electrodes 334 in order to define the temporal sequence, which is indicated by a series of arrows 961 connecting the selected electrodes in order. In at least some embodiments, the interface allows the selection of multiple electrodes 334 for any step. In at least some embodiments, the arrows 961 can include one or more graphical elements that indicate the individual steps by, for example, labeling (e.g., “step 1” or “1” for the first step and so on), use of different colors for the different steps, or the like or any combination thereof. In at least some embodiments, the interface also includes an arrangement 963 of stimulation parameter selections, such as electrode selection, stimulation amplitude, pulse width, or the like, which can be selected, input, or otherwise defined for the different steps represented by the individual arrows 961.

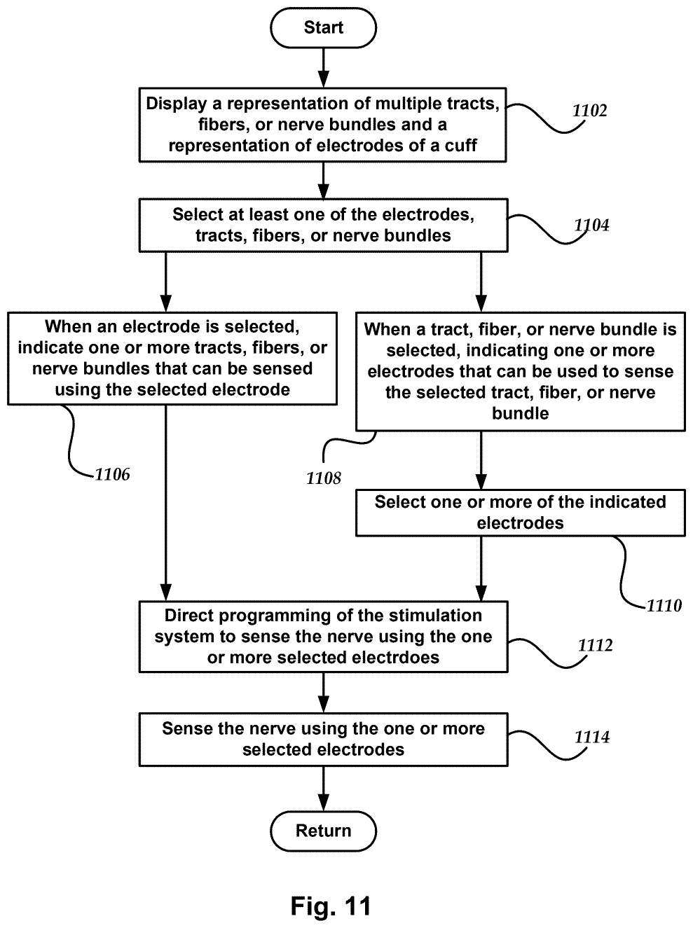

FIG. 10 illustrates another embodiment of an interface 1065 for defining or illustrating the temporal sequence of the electrodes 334 of the cuff 350. This embodiment includes, for each of the electrodes 334, a graph 1067 of amplitude (or an on/off activation state) over time indicating electrode activations 1069. As illustrated in FIG. 10, these graphs 1067 can illustrate a time sequence of electrode activations. In FIG. 10, the electrodes are activated in the following order: electrode 1-electrode 6-electrode 2-electrode 8-electrode 3 (which is also the same order of electrode activation illustrated in FIG. 9).

In at least some embodiments, the vertical axis of the graph indicates the amplitude of the current applied to the electrode. In at least some embodiments, the width of each electrode activation corresponds to the duration or pulse width of the activation. In at least some embodiments, two or more electrodes can be activated at any given time. In at least some embodiments, multiple electrodes can be activated at the same time and have different start times, stop times, or any combination thereof.

In at least some embodiments, a user can draw or modify the graphs 1067. In at least some embodiments, the interface 1065 includes controls for entering parameters, such as, for example, start time of an activation, stop time of an activation, the electrode(s) that are activated for a particular activation, an amplitude at each electrode that is activated, a duration of an activation, or the like or any combination thereof.

In at least some embodiments, the interface 1065 also includes an arrangement 1063 of stimulation parameter selections, such as electrode selection, stimulation amplitude, pulse start/stop time, pulse width, or the like, which can be selected, input, or otherwise defined for the different electrodes. The arrangement 1063 may be capable of defining multiple activations for each electrode.

In step 804, a representation of the nerve and, preferably, the electrodes of the cuff is displayed. FIG. 7 illustrates one embodiment of a display of the nerve and electrodes. FIG. 6 illustrates another embodiment of a display of the nerve, in the form of tracts, and electrodes.

In step 806, a time sequence of estimated stimulation fields, charge distributions, or activations of tracts, fibers, or nerve bundles corresponding to the temporal sequence of electrode activations is displayed. As described in more detail above, FIG. 7 illustrates a display of a stimulation field 341 arising from an activation of electrode 334a. A time sequence of estimate stimulation fields, like stimulation field 341, can be displayed with each of the estimated stimulation fields corresponding to one of the electrode activations arranged in the temporal/time sequence. With respect to FIG. 6, the time sequence of activations of tracts, fibers, or nerve bundles can include sequentially highlighting tract(s), fiber(s), or nerve bundle(s) that are activated by each electrode in the temporal sequence of electrode activations. In at least some embodiments, the time sequence of estimated stimulation fields, charge distributions, or activations of tracts, fibers, or nerve bundles can be presented as an animation or video. In at least some embodiments, the time sequence of estimated stimulation fields, charge distributions, or activations of tracts, fibers, or nerve bundles can be presented as a set of individual snapshots at different times. In at least some embodiments, the interface may allow for manually moving forward or backward along the time sequence.

In step 808, the user directs (or the system automatically initiates) programming of the stimulation system to stimulate the patient using the one or more selected electrodes. In at least some embodiments, prior to step 808, the user can modify the temporal sequence of electrode activations and then returning to step 806. In step 810, the stimulation system stimulates the patient using the one or more selected electrodes.

In at least some embodiments, the stimulation electrodes can also be used to sense electrical signals from the nerve. Examples of signals or the like that can be sensed include, but are not limited to, an evoked potential, evoked compound action potential (ECAP), tissue response, evoked resonant neural activity (ERNA), local field potential (LFP), ESG (electrospinogram), EEG (electroencephalogram), ECG (electrocardiogram), ECoG (electrocorticogram), or EMG (electromyogram) signal or the like or any combination thereof.

FIG. 11 is a flow chart of one embodiment of a method for selecting electrodes for sensing of a nerve of a patient using a cuff lead. In step 1102, a representation of multiple tracts, fibers, nerve bundles, or any combination thereof and a representation of the electrodes 334, 358a, 358b of a cuff 350 of a cuff lead (e.g., cuff lead 103 (FIG. 1)) is displayed (for example, on a display of a remote control, clinician programmer, or other suitable device or computer system, such as the system illustrated in FIG. 12 or devices/systems described in the reference cited herein). The displays or interfaces of FIGS. 5 and 6 can be used in the same manner as described for stimulation except that the objective is to identify electrodes for sensing signals or other electrical characteristics of one or more tracts, fibers, or nerve bundles 335. Any other suitable display can be used.

In step 1104, a user selects one (or more) of the electrodes 334, 358a, 358b; tracts, fibers, or nerve bundles 335. The method proceeds to either step 1106 or 1108 depending on the selection. Any suitable method can be used for making a selection can be used including, but not limited to, selection from a menu; point-and-click selection (or the like); alphanumerical entry of a designation for the electrode, tract, fiber, or nerve bundle; entry or selection of a symptom, disease, disorder, therapeutic effect, or the like followed by association with one or more tracts, fibers, or nerve bundles, or the like or any combination thereof.

In step 1106, when the user selects one (or more) of the electrodes 334, 358a, 358b, any of the tracts, fibers, or nerve bundles 335 that can be sensed using the selected electrode(s) are indicated on the display based on the relationship between the electrode(s) and the tracts, fibers, or nerve bundles. It will be understood that the method can return to step 1104 for another user selection.

In step 1108, when the user selects one (or more) of the tracts, fibers, or nerve bundles 335, any of the electrodes 334, 358a, 358b that can be used to sense electrical signals from the selected tract(s), fiber(s), or nerve bundle(s) are indicated on the display based on the relationship between the electrode(s) and the tracts, fibers, or nerve bundles. It will be understood that the method can return to step 1104 for another user selection. In step 1110, the user selects one or more of the indicated electrodes 334, 358a, 358b for sensing.

After step 1106 or 1110, in step 1112, the user directs (or the system automatically initiates) programming of the stimulation system to sense the nerve using the one or more selected electrodes. In step 1114, the system senses the nerve using the one or more selected electrodes. It will be understood that the flowcharts of FIGS. 4 and 11 can be integrated together to provide both stimulation and sensing using the electrodes of the cuff.

The flowchart of claim 8 and the interfaces of FIGS. 9 and 10 can also be used as described above except that, instead of a temporal sequence of electrode activations, there is a temporal sequence of electrode sensing using different electrodes. In at least some embodiments, the interfaces of FIGS. 9 and 10 can be used for a temporal sequence of electrode activations and electrode sensing.

FIG. 12 is a schematic overview of one embodiment of components of an electrical stimulation system 1204 that includes an implantable electrical stimulation arrangement 1200 with a lead 1202, stimulation circuitry 1206, a power source 1208, and an antenna 1210. The electrical stimulation system can be, for example, any of the electrical stimulation systems described above. It will be understood that the electrical stimulation system can include more, fewer, or different components and can have a variety of different configurations including those configurations disclosed in the stimulator references cited herein.

An external device, such as a clinician programmer (CP) or remote control (RC) 1207 (or any other suitable device or devices), can include a processor 1209, memory 1215, an antenna 1217, and a user interface 1211. The user interface 1211 can include, but is not limited to, a display screen on which a digital user interface can be displayed and any suitable user input device, such as a keyboard, touchscreen, mouse, track ball, or the like or any combination thereof.

Any power source 1208 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,1123, incorporated herein by reference in its entirety.

If the power source 1208 is a rechargeable battery or chargeable capacitor, the power source may be recharged/charged using the antenna 1210, if desired. Power can be provided for recharging/charging by inductively coupling the power source 1208 through the antenna 1210 to a recharging unit 1236 external to the user. Examples of such arrangements can be found in the references identified above.

In at least some embodiments, electrical current is emitted by the electrodes (such as electrodes 134 in FIG. 1) on the lead 1202 to stimulate nerve fibers, muscle fibers, or other body tissues near the electrical stimulation system. The stimulation circuitry 1206 can include, among other components, a processor 1234, a memory 1235, and a receiver 1232. The processor 1234 is generally included to control the timing and electrical characteristics of the electrical stimulation system. For example, the processor 1234 can, if desired, control one or more of the timing, frequency, strength, duration, and waveform of the pulses. In addition, the processor 1234 can select which electrodes can be used to provide stimulation, if desired. In some embodiments, the processor 1234 selects which electrode(s) are cathodes and which electrode(s) are anodes. In some embodiments, the processor 1234 is used to identify which electrodes provide the most useful stimulation of the desired tissue.

Any processor 1234 can be used and can be as simple as an electronic device that, for example, produces pulses at a regular interval or the processor can be capable of receiving and interpreting instructions from the CP/RC 1207 that, for example, allows modification of pulse characteristics. In the illustrated embodiment, the processor 1234 is coupled to a receiver 1232 which, in turn, is coupled to the antenna 1210. This allows the processor 1234 to receive instructions from an external source to, for example, direct the pulse characteristics and the selection of electrodes, if desired. Any suitable processor 1209 can be used for the CP/RC 1207.

Any suitable memory 1235, 1215 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In at least some embodiments, the antenna 1210 is capable of receiving signals (e.g., RF signals) from an antenna 1217 of a CP/RC 1207, which is programmed or otherwise operated by a user. The CP/RC 1207 can be a device that is worn on the skin of the user or can be carried by the user and can have a form similar to a pager, cellular phone, or remote control, if desired. As another alternative, the CP/RC 1207 may not be worn or carried by the user but may only be available at a home station or at a clinician's office.

The signals sent to the processor 1234 via the antenna 1210 and the receiver 1232 can be used to modify or otherwise direct the operation of the electrical stimulation system 1200. For example, the signals may be used to modify the pulses of the electrical stimulation system such as modifying one or more of pulse duration, pulse frequency, pulse waveform, and pulse strength. The signals may also direct the electrical stimulation system 1200 to cease operation, to start operation, t to start signal acquisition, to stop signal acquisition, to start charging the battery, or to stop charging the battery.

Optionally, the electrical stimulation system 1200 may include a transmitter (not shown) coupled to the processor 1234 and the antenna 1210 for transmitting signals back to the CP/RC 1207 or another unit capable of receiving the signals. For example, the electrical stimulation system 1200 may transmit signals indicating whether the electrical stimulation system 1200 is operating properly or not or indicating when the battery needs to be charged or the level of charge remaining in the battery. The processor 1234 may also be capable of transmitting information about the pulse characteristics so that a user or clinician can determine or verify the characteristics.

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification provides a description of the structure, manufacture, and use of the invention. Since many embodiments of the invention can be made without departing from the spirit and scope of the invention, the invention also resides in the claims hereinafter appended.

What is claimed as new and desired to be protected is:

1. A system for stimulating a nerve using a cuff lead disposed around the nerve, the cuff lead comprising a plurality of electrodes, the system comprising:
a programming device comprising
a display;
a memory having instructions stored thereon; and
a processor coupled to the display and the memory and configured to execute the instructions to perform actions, the action comprising:
displaying, on the display, a representation of a plurality of tracts, fibers, nerve bundles, modulation targets, symptoms, or any combination thereof and a representation of the electrodes;
in response to selection of at least one of the electrodes, displaying, on the display, an indication of which of the tracts, fibers, or nerve bundles can be activated or which of the modulation targets or symptoms can be affected, using the at least one selected electrode;
in response to selection of a one of the tracts, fibers, nerve bundles, modulation targets, or symptoms, displaying, on the display, an indication of at least one of the electrodes for activating the selected one of the tracts, fibers, or nerve bundles or for affecting the selected one of the modulation targets or symptoms; and
programming a stimulator to stimulate the nerve using the at least one selected electrode or the at least one indicated electrode.

2. The system of claim 1, further comprising
the cuff lead comprising a cuff body configured for disposition around the nerve with the plurality of electrodes disposed on the cuff body; and
the stimulator coupled or coupleable to the cuff lead and configured to generate stimulation signals and deliver the stimulation signals through at least one of the electrodes to stimulate the nerve.

3. The system of claim 1, wherein each modulation target comprises a body structure, the actions further comprising
displaying, on the display, a plurality of the body structures located on a representation of a body; and
in response to selection of a one of the body structures, displaying, on the display, an indication of which of the electrodes can, by stimulating the nerve, produce a therapeutic effect at the selected one of the body structures.

4. The system of claim 1, wherein the actions further comprise, in response to selection of the at least one of the electrodes and to obtaining at least a stimulation amplitude for each of the at least one of the electrodes, displaying, on the display, a representation of an estimated stimulation field or estimated charge distribution that can be generated using the selected at least one of the electrodes and the obtained simulation amplitude for each of the selected at least one of the electrodes.

5. The system of claim 1, wherein the representation of the electrodes comprises a representation of the cuff body unwrapped with the electrodes distributed thereon.

6. The system of claim 1, wherein the actions further comprise displaying, on the display, an image or graphical representation of the nerve.

7. The system of claim 1, wherein the actions further comprise receiving a series of selections of at least one of the electrodes to define a temporal sequence of electrode activations to stimulate the nerve.

8. The system of claim 7, wherein the actions further comprise displaying, on the display, a time sequence of activations of the tracts, fibers, or nerve bundles according to the temporal sequence of electrode activations.

9. The system of claim 7, wherein the actions further comprise displaying, on the display, a time sequence of estimated stimulation fields or estimated charge distributions generated according to the temporal sequence of electrode activations.

10. The system of claim 1, wherein displaying the indication of which of the tracts, fibers, or nerve bundles can be activated or which of the modulation targets or symptoms can be affected comprises selecting the indication to identify a characteristic of a relationship between the at least one selected electrode and the indicated tracts, fibers, nerve bundles, modulation targets, or symptoms.

11. The system of claim 10, wherein the characteristic comprises at least one of the following: a relative strength of the relationship, whether activation of the at least one selected electrode is beneficial or detrimental, or whether activation of the at least one selected electrode produces a neural activation effect or a neural blocking effect.

12. A system for stimulating a nerve using a cuff lead disposed around the nerve, the cuff lead comprising a plurality of electrodes, the system comprising:
a programming device comprising
a display;
a memory having instructions stored thereon; and
a processor coupled to the display and the memory and configured to execute the instructions to perform actions, the action comprising:
obtaining a stimulation program comprising a temporal sequence of electrode activations, wherein each of the electrode activations includes at least one electrode that is selected to be activated and a stimulation amplitude for each of the selected at least one electrode, wherein at least two of the electrode activations differ in electrode selection, stimulation amplitude of at least one of the at least one selected electrode, or any combination thereof;
displaying, on the display, a representation of the nerve and a representation of a plurality of the electrodes;
displaying, on the representation of the nerve on the display, a time sequence of estimated stimulation fields; estimated charge distributions; activations of tracts, fibers, or nerve bundles; or any combination thereof corresponding to the temporal sequence of electrode activations; and
programming the stimulator to stimulate the nerve using the temporal sequence of electrode activations.

13. The system of claim 12, wherein the actions further comprise displaying, on the display, the temporal sequence of electrode activations.

14. The system of claim 13, wherein displaying the temporal sequence of electrode activations comprises connecting individual representations of the electrodes according to the temporal sequence.

15. The system of claim 13, wherein displaying the temporal sequence of electrode activations comprises displaying, for each of multiple electrodes of the plurality of the electrodes, a graph of electrode activation or stimulation amplitude versus time according to the temporal sequence.

16. The system of claim 13, wherein obtaining the temporal sequence comprises receiving user input of stimulation parameters to define the temporal sequence of electrode activations.

17. The system of claim 13, wherein displaying the time sequence comprises displaying the time sequence as an animation or video.

18. A system for sensing a nerve using a cuff lead disposed around the nerve, the cuff lead comprising a plurality of electrodes, the system comprising:
a programming device comprising
a display;
a memory having instructions stored thereon; and
a processor coupled to the display and the memory and configured to execute the instructions to perform actions, the action comprising:
displaying, on the display, a representation of a plurality of tracts, fibers, or nerve bundles of the nerve and a representation of at least one or more of the electrodes;
in response to selection of at least one of the electrodes, displaying, on the display, an indication of which of the tracts, fibers, or nerve bundles can be sensed using the selected at least one of the electrodes;
in response to selection of a one of the tracts, fibers, or nerve bundles, displaying, on the display, an indication of at least one of the electrodes for sensing the selected one of the tracts, fibers, or nerve bundles; and
programming the stimulator to sense the nerve using the at least one selected electrode or the at least one indicated electrode.

19. The system of claim 18, further comprising displaying, on the display, an image or graphical representation of the nerve.

20. The system of claim 18, wherein the actions further comprise receiving a series of selections of at least one of the electrodes to define a temporal sequence of electrode sensing of the nerve.
